(19) United States
(12) Patent Application Publication
(10) Pub. No.: US 2025/0040983 A1
(43) Pub. Date: Feb. 6, 2025

(54) VESSEL HARVESTING DEVICE

(71) Applicants: TERUMO KABUSHIKI KAISHA, Tokyo (JP); TERUMO CARDIOVASCULAR SYSTEMS CORPORATION, Ann Arbor, MI (US)

(72) Inventors: Yusaku YAMAKI, Kanagawa (JP); Tatsunori FUJII, Kanagawa (JP)

(21) Appl. No.: 18/921,920

(22) Filed: Oct. 21, 2024

Related U.S. Application Data
(63) Continuation of application No. PCT/JP2023/015551, filed on Apr. 19, 2023.

(30) Foreign Application Priority Data
Apr. 21, 2022 (JP) 2022-070082

(51) Int. Cl. A61B 18/14; A61B 17/00; A61B 17/29; A61B 18/00

(52) U.S. Cl. CPC A61B 18/1447; A61B 2017/00778; A61B 2017/00969; A61B 2017/2936; A61B 2018/00077; A61B 2018/00083; A61B 2018/00589; A61B 2018/1455

(57) ABSTRACT

A vessel harvesting device includes: a cylindrical body extending along an axis; a jaw structure that is mounted on a distal end of the cylindrical body and has an upper jaw and a lower jaw that are opened and closed; and a cutter blade that is disposed between the upper jaw and the lower jaw and moves in a direction of the axis. The jaw structure has ridgelines formed at a distal end of the upper jaw and a distal end of the lower jaw, respectively. The upper jaw and the lower jaw have an asymmetric shape shifted in a first direction that is a width direction of the upper jaw and the lower jaw with respect to the direction of the axis of the cylindrical body. The positions of the ridgelines are offset from the central axis of the cylindrical body in the first direction.

### CROSS-REFERENCE TO RELATED APPLICATIONS

Pursuant to 35 U.S.C. § 119(e), this application is a continuation of International Application No. PCT/JP2023/015551, with an international filing date of Apr. 19, 2023, which claims the benefit of Japanese Patent No. 2022-070082 filed on Apr. 21, 2022, the contents of which are hereby incorporated by reference in their entireties.

### TECHNICAL FIELD

The present invention relates to a vessel harvesting device.

### BACKGROUND

In coronary artery bypass grafting (CABG), for example, a blood vessel harvested from a patient is connected so as to bypass a lesion site. The blood vessel to be used is harvested, for example, from the lower limb of the patient. An endoscopic vessel harvesting system (EVH system) is used to harvest a blood vessel.

An endoscopic vessel harvesting system includes an endoscope system, a pneumoperitoneum, a vessel dissection device, and a vessel harvesting device. The vessel is harvested in such a manner that the vessel dissection device is moved forward along the blood vessel while carbon dioxide gas is supplied by the pneumoperitoneum, and the blood vessel is dissected from surrounding fatty tissues. Thereafter, the vessel branches branching from the blood vessel are cut while hemostasis is performed by the vessel harvesting device. The vessel branches are cut while being observed with an endoscope. Thereafter, the vessel harvesting device is withdrawn, and the blood vessel is removed from the incision site, whereby the vessel harvesting is completed.

JP 2011-229923 A, for example, discloses a device that cuts a tissue under observation with an endoscope.

### SUMMARY

During vessel harvesting, surrounding tissues may not be sufficiently dissected in a vessel dissection operation using a vessel dissection device. In view of this, it is conceivable to impart a function of dissecting surrounding tissues to the vessel harvesting device in addition to a function of cutting a vessel branch. Therefore, it is conceivable to use the instrument disclosed in JP 2011-229923 A having a jaw structure with a distal end that is open wide.

However, the instrument disclosed in JP 2011-229923 A has a problem that an image of the vicinity of the distal end of the jaw structure is hardly captured by an imaging device disposed on the proximal side, and the procedure for dissecting surrounding tissues is hardly visually recognized.

An object of the present invention is to solve the problem described above.

One aspect of the following disclosure provides a vessel harvesting device including: a cylindrical body extending along an axis; a jaw structure that is mounted on a distal end of the cylindrical body and has an upper jaw and a lower jaw that are opened and closed; and a cutter blade that is disposed between the upper jaw and the lower jaw and moves in a direction of the axis along cutter grooves of the upper jaw and the lower jaw, the jaw structure having ridgelines formed at a distal end of the upper jaw and a distal end of the lower jaw, respectively, in which the upper jaw and the lower jaw have an asymmetric shape shifted in a first direction that is a radial direction with respect to the axis, and positions of the ridgelines are offset from the axis in the first direction.

Another aspect provides a vessel harvesting device including: a cylindrical body extending along an axis; a jaw structure that is mounted on a distal end of the cylindrical body and has an upper jaw and a lower jaw that are opened and closed; and a cutter blade that is disposed between the upper jaw and the lower jaw and moves in a direction of the axis along cutter grooves of the upper jaw and the lower jaw, in which the upper jaw and the lower jaw extend to the distal end while being inclined in a first direction that is a radial direction with respect to the axis, and distal portions of the upper jaw and the lower jaw located distal to the cutter grooves are within a region overlapping the axis.

In the vessel harvesting device according to the above aspects, an image of the vicinity of the distal end of the jaw structure is easily captured by the imaging device (endoscope) disposed on the proximal side, and thus, the procedure for dissecting surrounding tissues is facilitated.

### BRIEF DESCRIPTION OF DRAWINGS

FIG. 1 is a configuration diagram of a vessel harvesting system according to a first embodiment.

FIG. 2A is a side view of a distal end of a vessel harvesting device in FIG. 1 and a neighboring region thereof, and FIG. 2B is a perspective view illustrating a jaw structure in FIG. 2A from the distal end.

FIG. 3A is a plan view of an upper jaw in FIG. 2A as viewed from a clamping surface, and FIG. 3B is a plan view of a lower jaw in FIG. 2A as viewed from a clamping surface.

FIG. 4 is a side view illustrating the jaw structure that is removed from a cylindrical body.

FIG. 5A is a diagram illustrating the jaw structure that is closed as viewed from a first side surface, and FIG. 5B is a perspective view illustrating a cutter blade in a state where the jaw structure is removed from the cylindrical body.

FIG. 6A is a perspective view illustrating the clamping surface of the upper jaw, the clamping surface of the lower jaw, and the cutter blade in a state where the jaw structure is opened, and FIG. 6B is a side view illustrating the jaw structure in FIG. 6A that is closed.

FIG. 7A is a perspective view illustrating a state in which the cutter blade is retracted to the proximal side with the upper jaw being removed, and FIG. 7B is a perspective view illustrating a state in which the cutter blade protrudes to the distal side with the upper jaw being removed.

FIG. 8A is an explanatory diagram of a marking process of a vessel harvesting method, and FIG. 8B is an explanatory diagram of a process for dissecting a blood vessel with the vessel dissection device.

FIG. 9 is an explanatory diagram of a process for harvesting a blood vessel with the vessel harvesting device illustrated in FIG. 1.

FIG. 10A is a plan view illustrating a modification of the upper jaw in FIG. 3A, and FIG. 10B is a plan view illustrating a modification of the lower jaw in FIG. 3B.

### DESCRIPTION OF EXAMPLE EMBODIMENTS

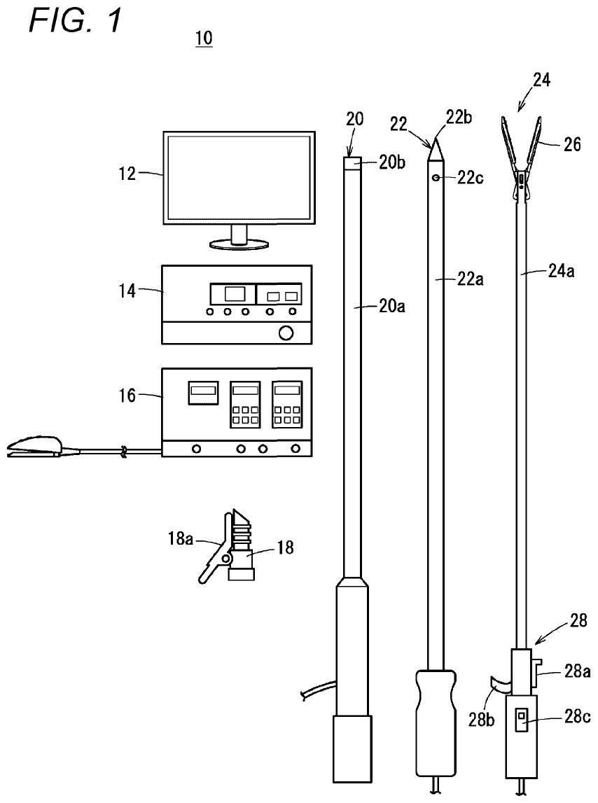

A vessel harvesting system 10 illustrated in FIG. 1 is an endoscopic vessel harvesting (EVH) system used for EVH. The vessel harvesting system 10 includes a display device 12, a high-frequency power source 14, a pneumoperitoneum apparatus 16, a trocar 18, an imaging device 20 (endoscope), a vessel dissection device 22, and a vessel harvesting device 24. Among them, the display device 12 is connected to the imaging device 20. The display device 12 displays an image captured by the imaging device 20. The high-frequency power source 14 supplies high-frequency power to the vessel harvesting device 24 to burn a tissue (a blood vessel 90 or a vessel branch 96). The pneumoperitoneum apparatus 16 supplies carbon dioxide gas to the vessel dissection device 22. The imaging device 20 includes a cylindrical body 20a and a camera 20b attached to a distal end of the cylindrical body 20a. The imaging device 20 is inserted into the body of the patient together with the vessel dissection device 22 or the vessel harvesting device 24 and captures an image of a treated site.

The trocar 18 is inserted into an incision site near a blood vessel. The trocar 18 facilitates introduction of the imaging device 20, the vessel dissection device 22, and the vessel harvesting device 24 into the body. The trocar 18 is fixed to the skin by a clip 18a.

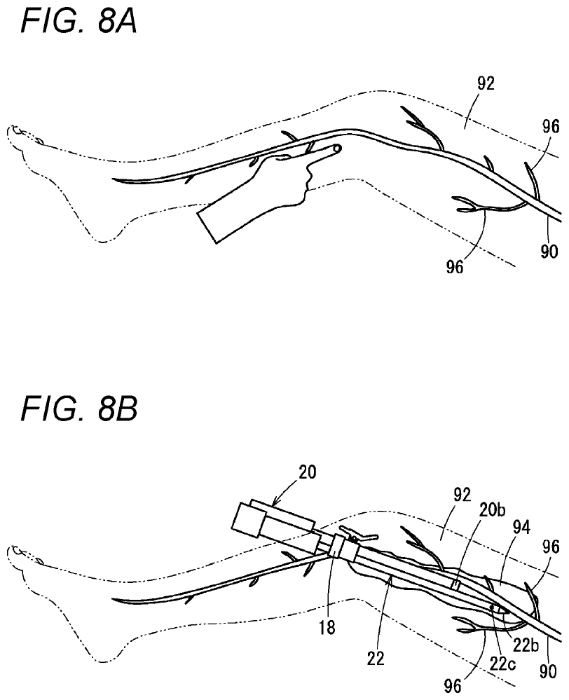

The vessel dissection device 22 includes a cylindrical body 22a and a conical dissection portion 22b attached to a distal end of the cylindrical body 22a. The cylindrical body 22a has an ejection hole 22c for releasing carbon dioxide gas in the vicinity of the distal end. The vessel dissection device 22 dissects the blood vessel 90 and a surrounding tissue 92 around the blood vessel 90 with the dissection portion 22b. The vessel dissection device 22 forms a cavity 94 around the blood vessel 90 by the carbon dioxide gas ejected from the ejection hole 22c (see FIG. 8B).

The vessel harvesting device 24 according to the present embodiment includes a cylindrical body 24a and a jaw structure 26 attached to a distal end of the cylindrical body 24a. The cylindrical body 24a is a cylindrical member extending in the direction of the axis, and accommodates a line (not illustrated) through which high-frequency power flows and an operation wire (not illustrated) or an operation rod (not illustrated) for operating the jaw structure 26.

The vessel harvesting device 24 cuts the vessel branch 96 of the blood vessel 90 that has been dissected by the jaw structure 26. The jaw structure 26 has a function of cutting the vessel branch 96 while burning the vessel branch and stopping bleeding with high-frequency power. The details of the jaw structure 26 will be described later.

The vessel harvesting device 24 has an operation hub 28 at a proximal portion. The operation hub 28 includes a cutter operation portion 28a, a jaw operation portion 28b, and an energization switch 28c. The cutter operation portion 28a performs an operation of moving a later-described cutter blade 34 in the direction of the axis. The jaw operation portion 28b performs an operation of opening and closing the jaw structure 26. The energization switch 28c switches between supply and stop of high-frequency power to the jaw structure 26. The jaw structure 26 specifically has the following configuration.

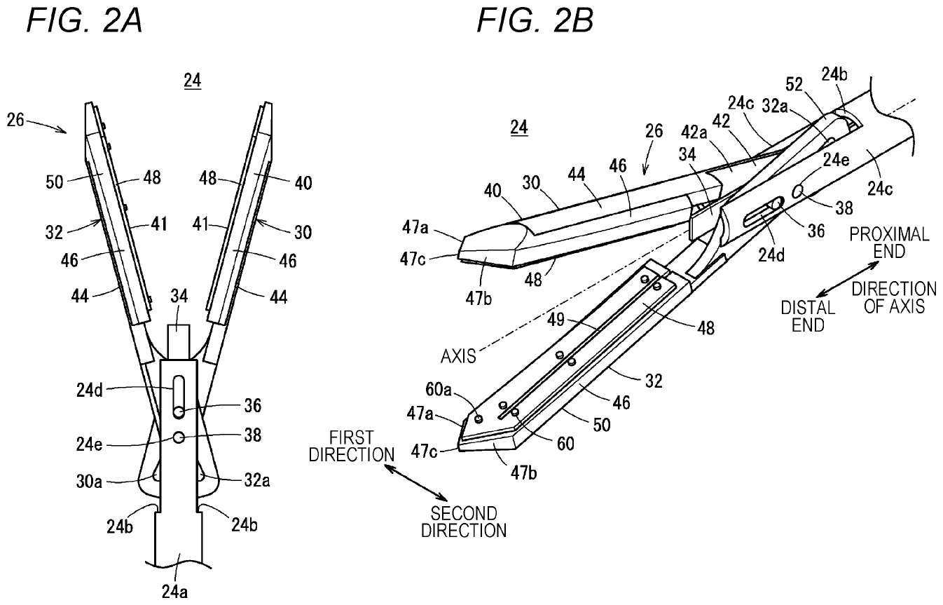

As illustrated in FIG. 2A, the jaw structure 26 is attached to the distal end of the cylindrical body 24a. As illustrated in FIG. 2B, the cylindrical body 24a has, on its distal portion, a pair of recessed grooves 24b formed by cutting out a part of the cylindrical body in the circumferential direction. The pair of recessed grooves 24b is formed at positions separated by 180° in the circumferential direction. Each of the recessed grooves 24b extends in the direction of the axis. The jaw structure 26 is accommodated in the recessed grooves 24b.

The cylindrical body 24a has a pair of support portions 24c extending toward the distal end between the pair of guide grooves 24d. The support portions 24c support the jaw structure 26. Each of the support portions 24c has a guide groove 24d and an opening/closing pin attachment hole 24e. The guide groove 24d is located distal to the opening/closing pin attachment hole 24e. The guide groove 24d extends in the direction of the axis. The opening/closing pin attachment hole 24e has a circular shape. The center positions of the guide groove 24d and the opening/closing pin attachment hole 24e are shifted by 90° in the circumferential direction of the cylindrical body 24a with respect to the center of the recessed groove 24b.

As illustrated in FIG. 2A, the jaw structure 26 includes an upper jaw assembly 30, a lower jaw assembly 32, and the cutter blade 34. The upper jaw assembly 30 and the lower jaw assembly 32 are connected using a shaft pin 36 and an opening/closing pin 38. The shaft pin 36 is fixed with respect to the upper jaw assembly 30 and the lower jaw assembly 32. The shaft pin 36 serves as a rotation center of the upper jaw assembly 30 and the lower jaw assembly 32. The shaft pin 36 is inserted into the guide grooves 24d of the cylindrical body 24a. The guide grooves 24d extend in the direction of the axis and allow the shaft pin 36 to move in the direction of the axis. The shaft pin 36 moves in the guide grooves 24d with displacement of the jaw structure 26 in the direction of the axis.

The opening/closing pin 38 is fixed to the cylindrical body 24a. The opening/closing pin 38 is displaced relative to the upper jaw assembly 30 and the lower jaw assembly 32 as the jaw structure 26 is displaced in the direction of the axis. As illustrated in FIGS. 2A and 2B, the opening/closing pin 38 is inserted into a first sliding groove 30a of the upper jaw assembly 30 and a second sliding groove 32a of the lower jaw assembly 32. When the jaw structure 26 moves forward or backward in the direction of the axis of the cylindrical body 24a, the opening/closing pin 38 slides in the first sliding groove 30a and the second sliding groove 32a. The upper jaw assembly 30 and the lower jaw assembly 32 rotate according to the position of the opening/closing pin 38 in the first sliding groove 30a and the second sliding groove 32a, and thus, the jaw structure 26 is opened and closed.

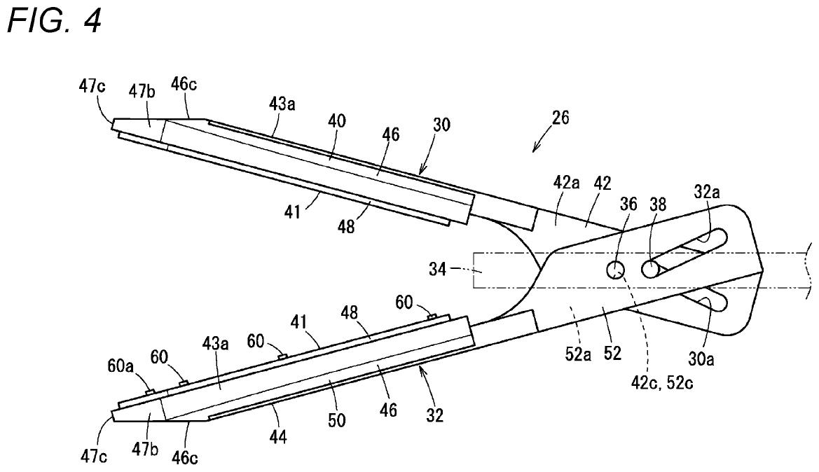

As illustrated in FIG. 4, the upper jaw assembly 30 includes an upper jaw 40 and a base 42. The upper jaw 40 is located on the distal side and has a clamping surface 41 orthogonal to the rotation direction. The base 42 is located proximal to the upper jaw 40 and is integrally connected to the upper jaw 40. The base 42 has a sliding surface 42a that is flat in a direction orthogonal to the clamping surface 41. The base 42 has a shaft hole 42c and the first sliding groove 30a. The shaft pin 36 is inserted into the shaft hole 42c. The shaft hole 42c is a rotation center of the upper jaw assembly 30. The first sliding groove 30a extends obliquely. The opening/closing pin 38 penetrates the first sliding groove 30a.

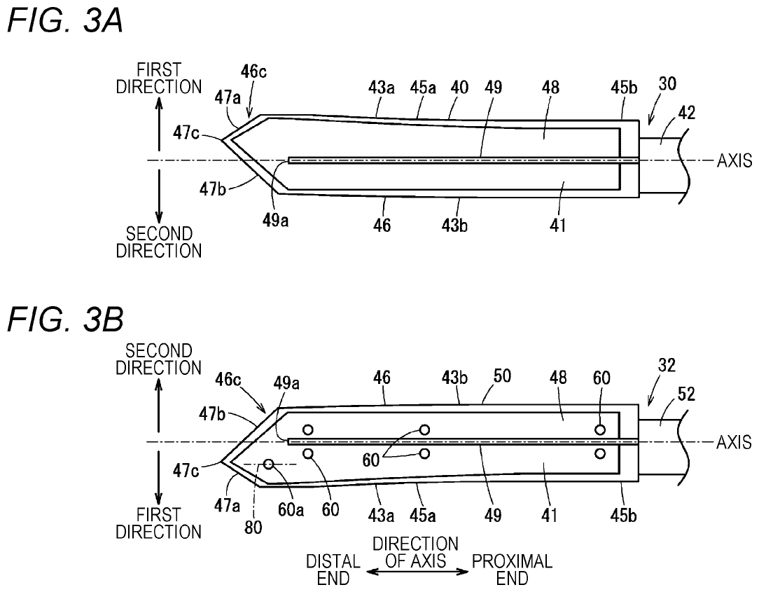

As illustrated in FIGS. 2B and 4, the upper jaw 40 includes a support body 44, a main body 46, and a planar electrode 48. As illustrated in FIG. 2B, the support body 44 is integrally connected to the base 42, and is formed of the same material (for example, metal) as the base 42. The support body 44 supports the main body 46. The main body 46 is formed of an insulating material such as resin. The main body 46 constitutes most of the upper jaw 40. As illustrated in FIG. 3A, the main body 46 extends while being slightly inclined with respect to the direction of the axis. The main body 46 is supported by the support body 44.

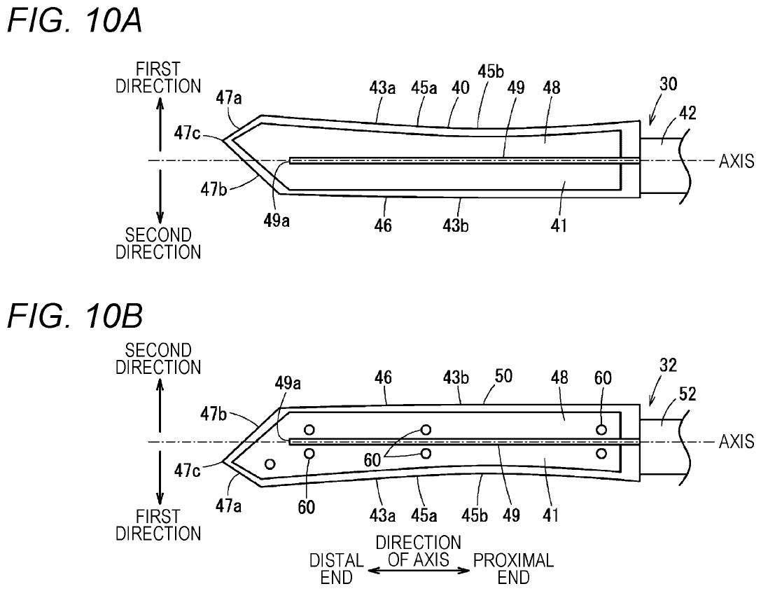

As illustrated in FIG. 3A, the main body 46 of the upper jaw 40 has a first side surface 43a in a first direction orthogonal to the axis, and has a second side surface 43b in a second direction opposite to the first direction. The center line of the main body 46 is inclined in the first direction with respect to the axis of the cylindrical body 24a. The first side surface 43a has a curved surface 45a having a gentle arc shape so as to project toward a cutter groove 49. The curved surface 45a of the first side surface 43a has a vertex 45b closest to the cutter groove 49 at the proximal portion. The second side surface 43b extends in parallel with the cutter groove 49. The position of the vertex 45b is not limited to the proximal portion of the curved surface 45a. As illustrated in FIGS. 10A and 10B, the upper jaw 40 and the lower jaw 50 may have vertexes 45b near intermediate portions of the curved surfaces 45a of the first side surfaces 43a in the direction of the axis.

The main body 46 has a distal portion 46c protruding from the support body 44 at a distal end thereof. The upper jaw 40 extends while being inclined in the first direction with respect to the axis, and thus, the distal portion 46c is shifted in the first direction with respect to the axis. The inclination of the upper jaw 40 is slight, so that a part of the distal portion 46c in the width direction overlaps with the axis of the cylindrical body 24a. Since the upper jaw 40 is slightly inclined, it is easy to concentrate the operation force on the distal portion 46c, and thus, it is possible to easily dissect a blood vessel from the surrounding tissues.

The distal portion 46c has a first inclined surface 47a and a second inclined surface 47b which are inclined with respect to the direction of the axis, and a ridgeline 47c. The first inclined surface 47a is a surface inclined in the first direction, and is adjacent to the first side surface 43a. The second inclined surface 47b is a surface inclined in the second direction, and is adjacent to the second side surface 43b. The ridgeline 47c is formed as a side where the first inclined surface 47a and the second inclined surface 47b meet. The ridgeline 47c is located at the distal end of the upper jaw assembly 30 and extends in a direction orthogonal to the clamping surface 41. The length of the first inclined surface 47a is shorter than the length of the second inclined surface 47b. The proximal end of the first inclined surface 47a is located distal to the proximal end of the second inclined surface 47b in the direction of the axis.

As illustrated in FIG. 3A, the first inclined surface 47a and the second inclined surface 47b meet at an acute angle at the ridgeline 47c. Due to the ridgeline 47c described above, the blood vessel can be preferably dissected from the surrounding tissues. In the upper jaw 40, the position of the ridgeline 47c at the distal end is offset from the direction of the axis in the first direction. In the upper jaw 40 in the present embodiment, the position of the ridgeline 47c is close to the position of the first side surface 43a, by which the visibility of the position where the dissection is performed is improved.

As illustrated in FIGS. 2A and 2B, the upper jaw 40 has the clamping surface 41 facing the lower jaw 50. As illustrated in FIG. 3A, the planar electrode 48 formed of a conductive material is disposed on the clamping surface 41. The planar electrode 48 is formed of a plate-like metal plate attached to the main body 46. The surface of the planar electrode 48 constitutes the clamping surface 41. The clamping surface 41 has the cutter groove 49 extending along the axis. The cutter groove 49 penetrates the planar electrode 48 and reaches the inside of the main body 46. The width of the cutter groove 49 is equal to or slightly larger than the thickness of the cutter blade 34. The cutter groove 49 extends along the axis of the cylindrical body 24a when the jaw structure 26 is closed. The cutter groove 49 guides the movement of the cutter blade 34 in the direction of the axis.

As illustrated in FIGS. 2B and 4, the lower jaw assembly 32 includes a lower jaw 50 and a base 52. The lower jaw 50 is located at a distal end of the base 52 and has the clamping surface 41 facing the upper jaw 40. The base 52 is located proximal to the lower jaw 50 and is integrally connected to the lower jaw 50. The base 52 has a sliding surface 52a that is flat in a direction orthogonal to the clamping surface 41. The sliding surface 52a slides on the sliding surface 42a of the upper jaw assembly 30. The base 52 has a shaft hole 52c and the second sliding groove 32a. The shaft pin 36 is inserted into the shaft hole 52c. The shaft hole 52c is a rotation center of the lower jaw assembly 32. The second sliding groove 32a extends obliquely in a direction opposite to the first sliding groove 30a. The opening/closing pin 38 penetrates the second sliding groove 32a.

As illustrated in FIGS. 2B and 3B, the lower jaw 50 includes a support body 44, a main body 46, a planar electrode 48, and a cutter groove 49. The lower jaw 50 has a vertically symmetrical shape with respect to the upper jaw 40, and thus, the detailed description of the shape is omitted. The components of the lower jaw 50 same as those of the upper jaw 40 are denoted by the same reference numerals. The lower jaw 50 includes a spacer 60 on the planar electrode 48. The spacer 60 protrudes from the surface (clamping surface 41) of the planar electrode 48.

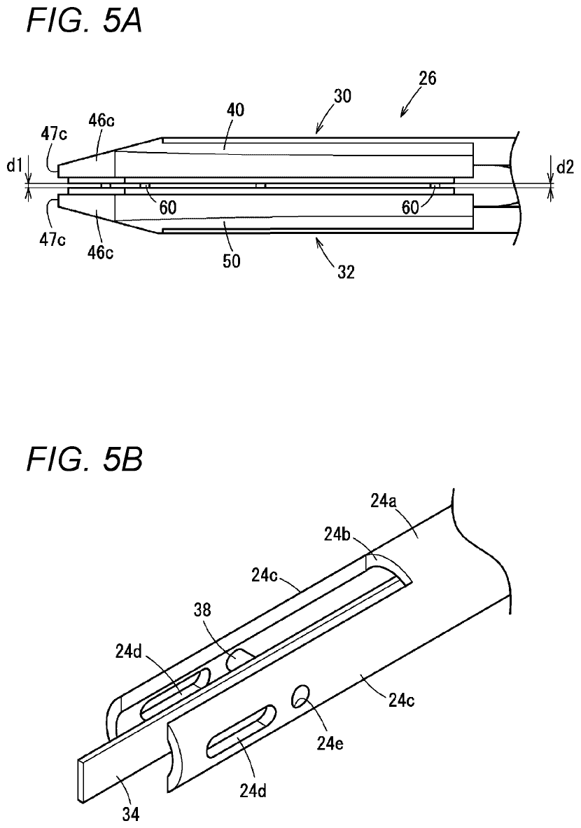

The spacer 60 protrudes from the planar electrode 48 at a height of, for example, 0.1 mm. The spacer 60 is, for example, a cylindrical insulating material. A plurality of spacers 60 is arranged at intervals in the extending direction of the cutter groove 49. As illustrated in FIG. 5A, the spacers 60 prevent contact between the planar electrode 48 of the upper jaw 40 and the planar electrode 48 of the lower jaw 50 to prevent short circuits when the jaw structure 26 is closed. In addition, the spacers 60 prevent cutting failure due to displacement of the blood vessel 90 or the vessel branch 96 when the cutter blade 34 protrudes along the cutter groove 49 to cut the blood vessel 90 or the vessel branch 96.

As illustrated in FIG. 3B, a distal spacer 60a that is the outermost spacer on the distal side among the plurality of spacers 60 is located distal to a distal-most portion 49a of the cutter groove 49 in the direction of the axis. The distal spacer 60a is located on a line 80 that passes through the ridgeline 47c and is parallel to the direction of the axis. The upper jaw 40 and the lower jaw 50 are inclined so as to be closed from the distal side as will be described later, so that the planar electrodes 48 are likely to come into contact with each other in a region closer to the distal end. The distal spacer 60a is disposed at the above place to prevent contact between the planar electrodes 48 at the distal portions of the upper jaw 40 and the lower jaw 50.

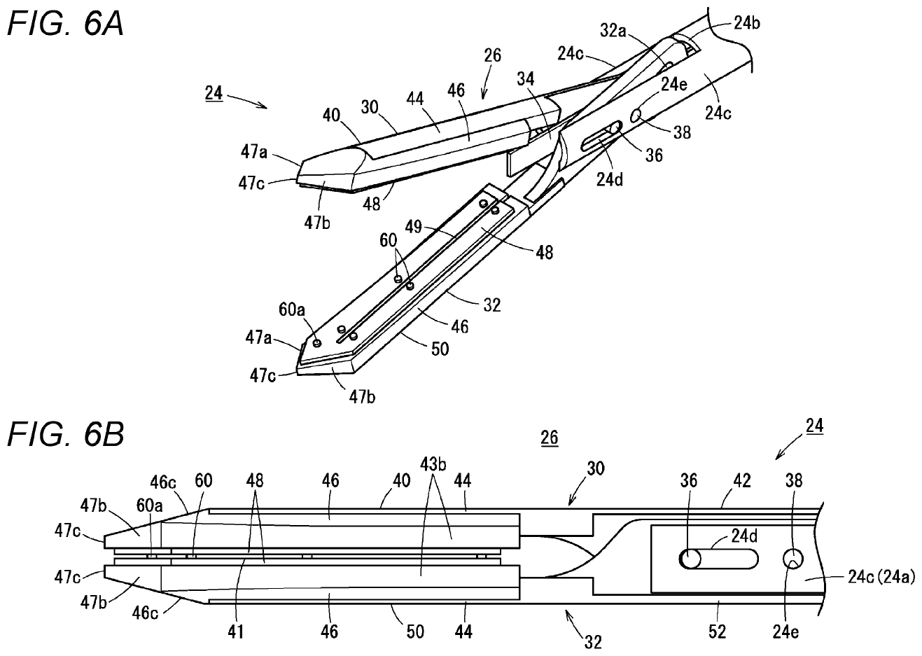

As illustrated in FIG. 4, the bases 42 and 52 of the upper jaw assembly 30 and the lower jaw assembly 32 are rotatably connected by the shaft pin 36 and the opening/closing pin 38. As illustrated in FIG. 6A, the cutter blade 34 is disposed between the base 42 of the upper jaw assembly 30 and the base 52 of the lower jaw assembly 32. The jaw structure 26 is displaceable in the direction of the axis with respect to the cylindrical body 24a. When the jaw structure 26 is located on the proximal side, the jaw structure 26 is opened, and the upper jaw 40 and the lower jaw 50 are separated as illustrated in FIG. 6A. When the jaw structure 26 is displaced to the distal side in the direction of the axis, the jaw structure 26 is closed as illustrated in FIG. 6B. The jaw structure 26 is moved using the jaw operation portion 28b of the operation hub 28 in FIG. 1.

As illustrated in FIG. 5A, in a state where the jaw structure 26 is closed, the clamping surface 41 of the upper jaw 40 and the clamping surface 41 of the lower jaw 50 have an inclination angle at which a clearance widens toward the proximal side. A clearance d1 between the upper jaw 40 and the lower jaw 50 at the distal end is smaller than a clearance d2 at the proximal end. Therefore, in the jaw structure 26, the upper jaw 40 and the lower jaw 50 are closed from the distal side.

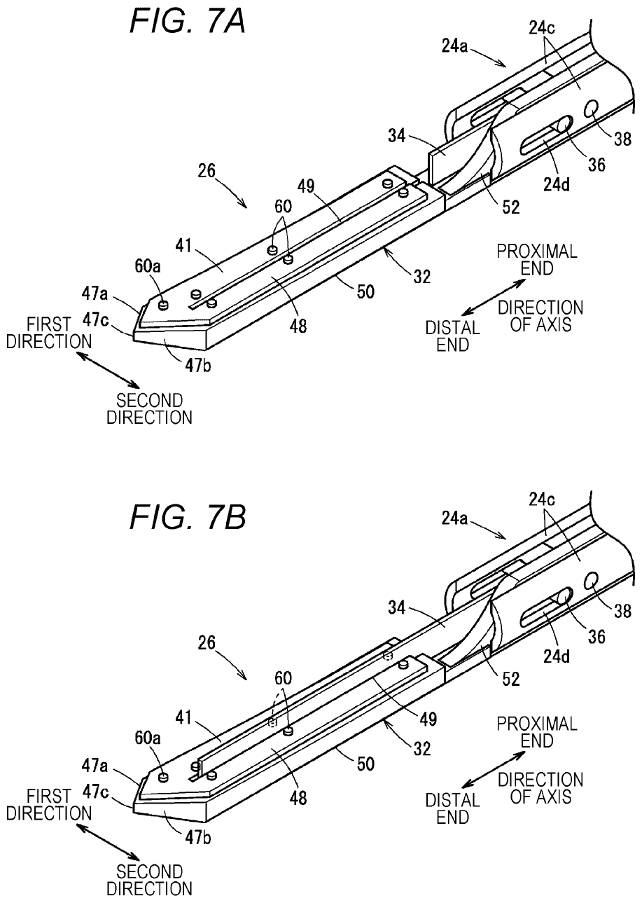

As illustrated in FIG. 5B, the cutter blade 34 extends in the direction of the axis of the cylindrical body 24a. The cutter blade 34 can protrude toward the distal end along the direction of the axis by the cutter operation portion 28a of the operation hub 28 illustrated in FIG. 1. The cutter blade 34 is biased toward the proximal side, and is positioned on the proximal side in an initial state as illustrated in FIG. 7A. When the cutter blade 34 is protruded with the jaw structure 26 closed, the cutter blade 34 protrudes along the cutter groove 49 toward the distal end in the direction of the axis as illustrated in FIG. 7B, and cuts the blood vessel 90 or the vessel branch 96 clamped by the jaw structure 26.

The vessel harvesting device 24 according to the present embodiment is configured as described above. The vessel harvesting system 10 is used for, for example, the following vessel harvesting method.

The vessel harvesting method includes a marking process as illustrated in FIG. 8A. This process includes a step for confirming the position of the saphenous vein at the tibia and a step for putting a mark of approximately 2.5 cm at the position below the knee joint.

Next, the vessel harvesting method proceeds to a process for inserting the trocar 18. During this process, the marked position is incised, and then, the trocar 18 is inserted. The trocar 18 is fixed to the skin by a clip 18a.

Next, the vessel harvesting method proceeds to a vessel dissection process as illustrated in FIG. 8B. During this process, the vessel dissection device 22 and the imaging device 20 are inserted through the trocar 18. This process includes an operation of dissecting the surrounding tissue 92 from the blood vessel 90 with the dissection portion 22b while imaging the blood vessel 90 with the camera 20b of the imaging device 20. The blood vessel 90 is dissected with the vessel dissection device 22 while carbon dioxide gas is ejected to the vicinity of the dissection portion 22b from the ejection hole 22c. Through this process, a cavity is formed around the blood vessel 90. After the blood vessel 90 in a predetermined region is dissected from the surrounding tissue, the vessel dissection device 22 and the imaging device 20 are removed from the body.

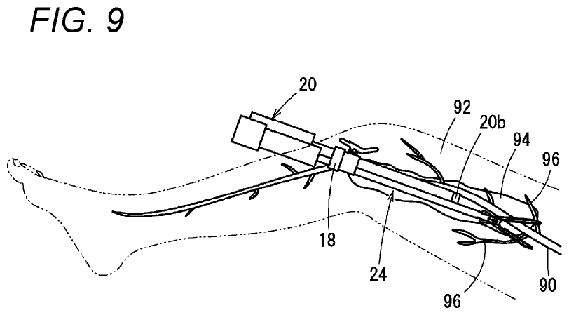

Next, the vessel harvesting method proceeds to a vessel harvesting process as illustrated in FIG. 9. The vessel harvesting process is performed using the vessel harvesting device 24. This process includes a step for cutting the vessel branch 96 with the vessel harvesting device 24. The vessel harvesting device 24 and the imaging device 20 are inserted into the cavity around the blood vessel 90 through the trocar 18. The imaging device 20 is placed on the proximal side with respect to the vessel harvesting device 24 and captures an image of the jaw structure 26 of the vessel harvesting device 24 from the proximal side.

The process for cutting the vessel branch 96 using the vessel harvesting device 24 is performed by the following steps. First, a step for placing the opened jaw structure 26 at the position of the vessel branch 96 under observation with the imaging device 20 is performed. The jaw structure 26 is then closed to clamp the vessel branch 96 between the upper jaw 40 and the lower jaw 50. Then, a step for supplying high-frequency power to the vessel harvesting device 24 is performed. High-frequency power is supplied between the planar electrode 48 of the upper jaw 40 and the planar electrode 48 of the lower jaw 50, and the clamped vessel branch 96 is burned to stop bleeding. Next, a step for cutting the vessel branch 96 is performed by advancing the cutter blade 34 along the cutter groove 49.

Thereafter, an operation of further advancing the vessel harvesting device 24 to cut another vessel branch 96 is performed. In the vessel harvesting device 24 according to the present embodiment, the ridgelines 47c appear at the distal end when the jaw structure 26 is closed. Therefore, during the vessel harvesting process, when a region where the surrounding tissue 92 is not sufficiently dissected is found in a part of the blood vessel 90, the surrounding tissue 92 can be dissected using the ridgelines 47c. The jaw structure 26 is shifted in the first direction with respect to the direction of the axis of the cylindrical body 24a, and the ridgelines 47c are offset from the direction of the axis. Therefore, the vessel harvesting device 24 makes it possible to visually recognize the state near the distal end of the jaw structure 26 with the imaging device 20 located on the proximal side. In addition, the first side surface 43a curved so as to protrude toward the cutter groove 49 further improves the visibility of the vicinity of the distal end by the imaging device 20. In this manner, the vessel harvesting device 24 facilitates the dissection of the remaining surrounding tissue 92.

After cutting of the vessel branch 96 and the blood vessel 90 in the desired region is completed, the vessel harvesting device 24 and the imaging device 20 are withdrawn from the patient's body. Thereafter, the blood vessel 90 is removed from the incision site, whereby the vessel harvesting method is completed.

The vessel harvesting device 24 according to the present embodiment described above is summarized below.

One aspect provides a vessel harvesting device 24 including: a cylindrical body 24a extending along an axis; a jaw structure 26 that is mounted on a distal end of the cylindrical body and has an upper jaw 40 and a lower jaw 50 that are opened and closed; and a cutter blade 34 that is disposed between the upper jaw and the lower jaw and moves in a direction of the axis along cutter grooves 49 of the upper jaw and the lower jaw, the jaw structure having ridgelines 47c formed at a distal end of the upper jaw and a distal end of the lower jaw, respectively, in which the upper jaw and the lower jaw have an asymmetric shape shifted in a first direction that is a radial direction with respect to the axis, and positions of the ridgelines are offset from the axis in the first direction.

The above-described vessel harvesting device makes it possible to easily confirm the state in the vicinity of the distal end of the jaw structure with the imaging device, and thus, can facilitate the operation of dissecting tissues left around the blood vessel.

In the vessel harvesting device described above, the upper jaw and the lower jaw may each have a first inclined surface 47a adjacent to the ridgeline in the first direction and a second inclined surface 47b adjacent to the ridgeline in a second direction opposite to the first direction, the ridgeline being an intersection line between the first inclined surface and the second inclined surface. The vessel harvesting device described above has sharp ridgelines at the distal end, thereby making it easy to dissect the surrounding tissue from the blood vessel.

In the vessel harvesting device, the first inclined surfaces and the second inclined surfaces may meet at the ridgelines at an acute angle. The vessel harvesting device having such ridgelines makes it easy to dissect the surrounding tissue from the blood vessel.

In the above-described vessel harvesting device, the upper jaw and the lower jaw may each have a first side surface 43a adjacent to the first inclined surface on a proximal side, the first side surface having a curved surface 45a that protrudes to the axis along an arc. In the vessel harvesting device, the first side surface does not interfere with the imaging of the distal end of the jaw structure from the proximal end, whereby visibility near the distal end is improved.

In the above-described vessel harvesting device, each of the first side surfaces may approach the axis as the first side surface extends to a proximal end, and the arc may have a vertex 45b located at the proximal end of the first side surface. The vessel harvesting device has excellent visibility near the distal end.

In the above-described vessel harvesting device, the curved surface of each of the first side surfaces may have a vertex closest to the axis at an intermediate portion in the direction of the axis. The vessel harvesting device has excellent visibility near the distal end.

In the above-described vessel harvesting device, the upper jaw and the lower jaw may respectively have planar electrodes 48 formed of a conductive material on surfaces facing each other, and at least one of the planar electrode of the upper jaw and the planar electrode of the lower jaw may have a spacer 60 formed of an insulating material and protruding from the planar electrode. The vessel harvesting device can prevent short circuits between the planar electrodes and supply sufficient high-frequency power to the blood vessel, enabling reliable hemostasis.

In the above-described vessel harvesting device, the upper jaw and the lower jaw may have an inclination angle at which a clearance between the surfaces facing each other widens toward the proximal end when the jaw structure is closed.

Another aspect provides a vessel harvesting device including: a cylindrical body extending along an axis; a jaw structure that is mounted on a distal end of the cylindrical body and has an upper jaw and a lower jaw that are opened and closed; and a cutter blade that is disposed between the upper jaw and the lower jaw and moves in a direction of the axis along cutter grooves of the upper jaw and the lower jaw, in which the upper jaw and the lower jaw extend to the distal end while being inclined in a first direction that is a radial direction with respect to the axis, and distal portions of the upper jaw and the lower jaw located distal to the cutter grooves are within a region overlapping the axis.

In the above-described vessel harvesting device, the distal ends of the upper jaw and the lower jaw are slightly curved within a region overlapping the axis, whereby it is easy to concentrate the operation force on the distal ends, and it is possible to easily dissect the surrounding tissue from the blood vessel. In addition, since the jaw structure is curved, it is easy to visually recognize the operation site at the distal end from the imaging device on the proximal side.

Note that the present invention is not limited to the above-described embodiment, and various configurations can be adopted without departing from the gist of the present invention.

1. A blood vessel harvesting device comprising:
a cylindrical body extending along an axis;
a jaw structure coupled to a distal end portion of the cylindrical body and comprising a first jaw and a second jaw that are openable to an open state and closeable to a closed state; and
a cutter blade disposed between the first jaw and the second jaw, the cutter blade being moveable in a direction of the axis along cutter grooves defined by the first jaw and the second jaw,
the jaw structure having ridgelines formed at a distal-most tip end of the first jaw and a distal-most tip end of the second jaw,
wherein the ridgelines are offset from the axis in a first direction such that the first jaw and the second jaw have asymmetric shapes with respect to the axis.

2. The blood vessel harvesting device of claim 1, wherein the first jaw and the second jaw each have a first inclined surface adjacent to the ridgeline and extending in the first direction and a second inclined surface adjacent to the ridgeline and extending in a second direction opposite to the first direction, the ridgelines being an intersection line between the first inclined surface and the second inclined surface.

3. The blood vessel harvesting device of claim 2, wherein the first inclined surfaces and the second inclined surfaces meet at the ridgelines and define an acute angle therebetween.

4. The blood vessel harvesting device of claim 2, wherein the first jaw and the second jaw each have a first side surface adjacent to the first inclined surface and extending proximally, the first side surface comprising a curved surface that defines an arc having a center located away from the axis in the first direction.

5. The blood vessel harvesting device of claim 4, wherein each of the first side surfaces approaches the axis as the first side surface extends to a proximal end thereof where the first side surfaces are closest to the axis.

6. The blood vessel harvesting device of claim 4, wherein the curved surface of each of the first side surfaces is closest to the axis at an intermediate portion of the curved surface.

7. The blood vessel harvesting device of claim 1, wherein the first jaw and the second jaw each have a planar electrode formed of an electrically conductive material on surfaces facing each other, and at least one of the surfaces has a spacer formed of an electrically insulating material that is protruding from the planar electrode.

8. The blood vessel harvesting device of claim 1, wherein the first jaw and the second jaw each have a planar electrode on surfaces facing each other, and wherein while the jaw structure is in the closed state the planar electrodes of the first jaw and the second jaw define a non-zero inclination angle therebetween.

9. A blood vessel harvesting device comprising:
a cylindrical body extending along an axis;
a jaw structure coupled to a distal end portion of the cylindrical body and comprising a first jaw and a second jaw that are openable to an open state and closeable to a closed state; and
a cutter blade disposed between the first jaw and the second jaw, the cutter blade being moveable in a direction of the axis along cutter grooves defined by the first jaw and the second jaw,
wherein, while the jaw structure is in the closed state: (i) opposing face surfaces of the first jaw and the second jaw are inclined in relation to each other and in relation to the axis, and (ii) the axis extends between portions of the first jaw and the second jaw that are located distal to the cutter grooves.

10. The blood vessel harvesting device of claim 9, wherein, while the jaw structure is in the closed state, a clearance between the opposing face surfaces of the first jaw and the second jaw at a distal end of the jaw structure is smaller than a clearance at a proximal end of the jaw structure.

11. The blood vessel harvesting device of claim 9, wherein a distal-most tip end of the first jaw and a distal-most tip end of the second jaw are each offset from the axis in a first direction such that the first jaw and the second jaw have asymmetric shapes with respect to the axis.

12. The blood vessel harvesting device of claim 9, wherein the opposing face surfaces of the first jaw and the second jaw each comprise a planar electrode formed of an electrically conductive material.

13. The blood vessel harvesting device of claim 12, wherein at least one of the opposing face surfaces has one or more spacers formed of an electrically insulating material that is protruding from the planar electrode.
